(19) United States
(12) Patent Application Publication
(10) Pub. No.: US 2023/0074733 A1
(43) Pub. Date: Mar. 9, 2023

(54) GEL NAIL STICKER CONTAINING GRAPHENE AND MANUFACTURING METHOD THEREOF

(71) Applicant: TRANSURFING Co., Ltd, Seoul (KR)

(72) Inventor: Shin Woong KOH, Seoul (KR)

(21) Appl. No.: 17/558,386

(22) Filed: Dec. 21, 2021

(30) Foreign Application Priority Data
Aug. 19, 2021 (KR) 10-2021-0109134

(51) Int. Cl. A45D 29/00; A61K 8/02; A61K 8/19; A61Q 3/02

(52) U.S. Cl. CPC A45D 29/001; A61K 8/0233; A61K 8/19; A61Q 3/02

(57) ABSTRACT

The present invention relates to a gel nail sticker containing graphene and a manufacturing method thereof, and more particularly, to a gel nail sticker in which some layers of a laminate body forming the gel nail sticker contain graphene, so that the gel nail sticker has improved retention force, anti-bacterial properties, anti-fungal properties, and the like, and a manufacturing method thereof. Specifically, the gel nail sticker of the present invention is a laminate body in which an adhesive layer containing graphene and an adhesive, a print layer containing a pigment material, and a transparent coating layer containing a curing resin are sequentially laminated.

### CROSS-REFERENCE TO RELATED APPLICATION

This application claims priority to Korean Patent Application No. 10-2021 0109134 filed on Aug. 19, 2021 and all the benefits accruing therefrom under 35 U.S.C. § 119, the contents of which are incorporated by reference in their entirety.

### BACKGROUND

The present disclosure relates to a gel nail sticker containing graphene and a manufacturing method thereof, and more particularly, to a gel nail sticker in which some layers of a laminate body forming the gel nail sticker contain graphene, so that the gel nail sticker has improved durability, retention force, adhesive force, and the like, and a manufacturing method thereof.

In the typical general nail art, a method of applying liquid pigments such as nail polish of various colors to nails and toenails, followed by drying the liquid pigments has been widely used.

However, the method of applying liquid pigments as described above requires a long period of time for drying the pigments after the application thereof, which causes inconvenience to users. Particularly, in the typical nail art, it is not easy for the users to draw patterns themselves, so that the users separately pay a high cost to professionals for their services in most cases. However, various shapes, letters, or the like printed on nails are erased without going for several days due to frequent hand washing, which has raised a problem in that the effectiveness of the services is not sufficient over high-cost expenditures.

Based on the fact, gel nail stickers which can be attached to nails have recently been released and used. Unlike the method of using and applying liquid pigments, gel nail stickers have the advantage in that stickers with various patterns according to a consumer's preference may be easily attached on and detached from nails or toenails.

However, since gel nail stickers are cured and used in a rigid shape, there is a problem in that the gel nail stickers cause a foreign body sensation, and are prone to scratches and imprints when used in real life, which reduces durability. Also, the gel nail stickers are rigid, and thus, have a low retention force, so that it is difficult to attach the gel nail stickers on curved nails of an ordinary person.

Furthermore, gel nail stickers are simply a sticker, and thus, are vulnerable to wear and tear, and when used for a long period of time, bacteria may grow. Also, there is a problem in that users feel frustrated because of the poor air permeability of the gel nail stickers.

As another type of nail art, artificial nails with excellent durability have also been developed, but the artificial nails lack retention force and elasticity. As a result, due to poor fit and adhesion, there is a disadvantage in that artificial nails do not naturally settle on nails, and thus, less aesthetic, and come off entirely from the nails.

Numerous papers and patent documents are referenced throughout this specification and their citations are indicated. The disclosure contents of the cited papers and patent documents are incorporated herein by reference in their entirety to more clearly describe the level of the technical field to which the present invention pertains and the content of the present invention.

### PRIOR ART DOCUMENT

### Patent Document

(Patent Document 1) Korean Patent Laid-Open Publication No. 10-1728432 (Published on Apr. 13, 2017)

(Patent Document 2) Korean Patent Laid-Open Publication No. 10-1240508 (Published on Mar. 28, 2013)

(Patent Document 3) Korean Patent Laid-Open Publication No. 10-2017-0018649 (Published on Feb. 20, 2017)

### SUMMARY

The present invention has been designed to solve the above-described typical limitations, and the purpose of the present invention is as follows.

The present disclosure provides a gel nail sticker and a manufacturing method thereof, wherein the gel nail sticker contains graphene in at least one layer of a laminate body forming the gel nail sticker, and thus, has improved durability, retention force, and adhesive force.

The present disclosure also provides a gel nail sticker and a manufacturing method thereof, wherein the gel nail sticker contains graphene in some layers of a laminate body forming the gel nail sticker, and thus, has improved anti-bacterial/anti-fungal properties and air permeability to be used eco-friendly by preventing bacteria from growing even when the gel nail sticker is used for a long period of time.

The present disclosure also provides a gel nail sticker and a manufacturing method thereof, wherein the gel nail sticker contains graphene in some layers of a laminate body forming the gel nail sticker, and thus, may achieve deep gloss which is unique to gel nails.

The tasks of the present invention are not limited to the above-mentioned tasks, and other tasks not mentioned will be clearly understood by those skilled in the art from the following descriptions.

In accordance with an exemplary embodiment of the present invention, a gel nail sticker includes a laminate body in which an adhesive layer containing an adhesive component, a print layer containing a pigment for expressing colors, and a transparent coating layer containing a curing resin are sequentially laminated, wherein at least one layer of the laminate body contains graphene.

In a preferred embodiment of the present invention, the adhesive layer may contain graphene particles.

In accordance with another exemplary embodiment of the present invention, a method for manufacturing of gel nail sticker includes an adhesive layer forming step S10 of preparing an adhesive composition in which a resin/binder and a solvent are mixed to form an adhesive layer, a print layer forming step S20 of applying a composition containing a pigment for expressing colors on top of the adhesive layer to form a print layer, and a transparent coating layer forming step S30 of forming a transparent coating layer containing a curing resin on top of the print layer.

In a preferred embodiment of the present invention, the adhesive layer may contain graphene particles.

In a preferred embodiment of the present invention, the graphene particles contained in a portion of the laminate body may be contained in approximately 0.005 mass % to 0.1 mass %, preferably approximately 0.007 mass % to 0.05 mass % based on the total mass of a composition forming each layer.

In a preferred embodiment of the present invention, the graphene particles contained in the laminate body may have a particle diameter of approximately 10 nm to 100 nm, preferably approximately 50 nm to 80 nm.

### BRIEF DESCRIPTION OF THE DRAWINGS

Exemplary embodiments can be understood in more detail from the following description taken in conjunction with the accompanying drawings, in which:

FIG. 1 is a view illustrating the shape of graphene included in a portion of a laminate body forming a gel nail sticker in accordance with an exemplary embodiment of the present invention;

FIG. 2 is a view illustrating one example of the cross-section of a gel nail sticker in accordance with another exemplary embodiment of the present invention;

FIG. 3 is a flowchart illustrating one example of a method for manufacturing a gel nail sticker in accordance with still another exemplary embodiment of the present invention; and

FIG. 4 shows that a gel nail sticker is more glossy when containing graphene in accordance with an exemplary embodiment of the present invention, due to the refection effect of the graphene.

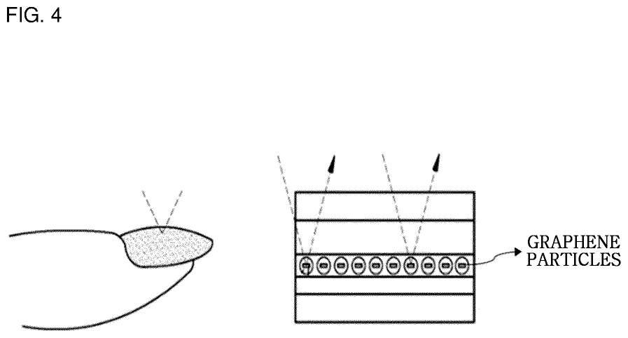

### DETAILED DESCRIPTION OF EMBODIMENTS

Hereinafter, a gel nail sticker and a manufacturing method thereof in accordance with an exemplary embodiment of the present invention will be described in detail with reference to the accompanying drawings.

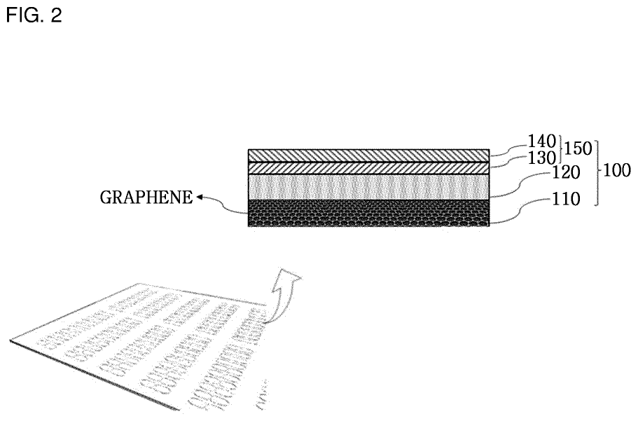

FIG. 2 is a view illustrating one example of the cross-section of a gel nail sticker in accordance with an exemplary embodiment of the present invention.

Referring to FIG. 2, a gel nail sticker 100 of the present invention is characterized by being a laminate body in which an adhesive layer 110, a print layer 120, and a transparent coating layer 150 are sequentially laminated.

In an embodiment of the present invention, at least one layer of the adhesive layer 110, the print layer 120, and the transparent coating layer 150 may contain graphene.

In an embodiment of the present invention, it is preferable that the adhesive layer 110 contains graphene.

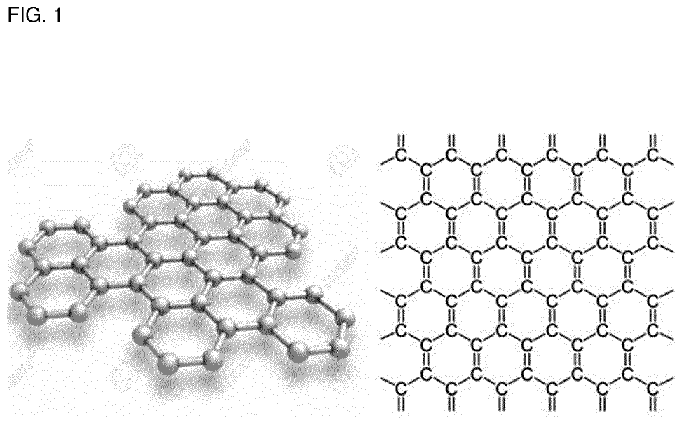

Graphene, which is contained in a portion of the laminate body of the present invention, is one of allotropes of carbon and has a structure in which carbon atoms are gathered to form a two-dimensional plane. The carbon atoms forms a hexagonal lattice wherein a carbon atom is positioned at vertices of the hexagon (see FIG. 1). The above shape is also referred to as a honeycomb structure or honeycomb lattice. Graphene is a thin film with a thickness of one atom, and is well known to have high physical and chemical stability while being extremely thin with a thickness of approximately 0.2 nm (1 nm is 1 billionth of a meter), that is, approximately 2 billionths of a meter.

Particularly, the strength of graphene is approximately 200 times or greater than that of steel, the thermal conductivity thereof is approximately 2 times or greater than that of diamond, which boasts the best thermal conductivity, and the elasticity thereof is also excellent, so that graphene is known to maintain its original nature even when stretched or bent. The inventors of the present invention paid attention to such properties of graphene, and found that when graphene was contained in some layers of the nail art of the present invention, the nail art has improved anti-bacterial properties, anti-fungal properties, composition, retention force, as well as adhesive force.

Particularly, graphene of the present invention has the effect of suppressing the growth of pseudomonas aeruginosa, which is easily found in a nail when a nail sticker is used for a long period of time.

Meanwhile, as described above, it is preferable that graphene is contained in an adhesive layer, which is a layer in direct contact with the nail. This is to maximize the anti-bacterial/anti-fungal effect since the growth of bacteria is more active in the adhesive layer in contact with a human body than between layers forming a laminate body.

The adhesive layer may be completed by a coating composition in which graphene particles, a resin/binder, and a solvent are mixed. Here, graphene particles may be contained in a range of approximately 0.005 mass % to 0.1 mass %, preferably approximately 0.007 mass % to 0.05 mass %, based on the total mass of the adhesive layer. Various types of typical resins/binders may be applied to the present embodiment, and depending on an embodiment, the binder may be composed of a transparent material or a material having a color or a predetermined transmittance.

Meanwhile, in another embodiment of the present invention, the adhesive layer may further contain a silver nano-material to further enhance antibacterial properties.

In an embodiment of the present invention, the resin/binder may be one or more selected from the group consisting of Hema/Di-Hema-trimethylhexyl dicarbamate and Trimethylolpropane trimethacrylate.

The content of the resin/binder may be appropriately adjusted by a person skilled in the art such that a corresponding layer satisfies 100 mass %.

When graphene was contained in the adhesive layer in the above-described range, it was confirmed that durability, adhesive force, and retention force were excellent, and when out of the above-described range, the physical properties of graphene itself were enhanced, so that adhesive force and retention force were lowered.

Particularly, graphene particles contained in the adhesive layer may be included as graphene particles having a particle diameter of approximately 10 nm to 100 nm, preferably approximately 50 nm to 80 nm. When the graphene particles has a size within the above range, the adhesive force is greatly improved, which is presumed that when the diameter of the graphene particles is formed within the above range, the attractive force between the particles and the attractive force with the adhesive layer is greater than the repulsive force, so that the adhesive force is improved.

In an embodiment of the present invention, the adhesive layer 110 may contain an adhesive component. The adhesive layer 110 is a portion directly attached to a nail or toenail, which is a layer that is cured while being dried at room temperature, and is formed of a soft material to correspond to the curved surface of the nail or toenail. The adhesive layer 110 may be formed to a thickness of approximately 10 μm to 50 μm.

In an embodiment of the present invention, the print layer 120 is a layer which uses pigments with various colors to express a desired color, pattern, or transmittance, and is a layer which maintains softness. That is, the print layer 120 may express a background color and/or a background pattern and/or transmittance of the gel nail sticker. The print layer 120 may be formed to a thickness of approximately 20 μm to 100 μm.

In an embodiment of the present invention, the transparent coating layer 150 may be composed of one layer or a plurality of layers 130 and 140 of two or more layers.

In an embodiment of the present invention, the transparent coating layer 150 includes an ultraviolet (UV) curing resin and/or a heat curing resin. The transparent coating layer 150 may be formed to a thickness of approximately 50 μm to 200 μm.

The ultraviolet curing resin of the transparent coating layer 150 may be any one of urethane acrylate, epoxy acrylate, and polyester acrylate, or a mixture of at least one thereof.

According to an embodiment of the present invention, the transparent coating layer 150 may further include a photoinitiator. The photoinitiator is a raw material which absorbs energy from ultraviolet light, and which causes a photopolymerization reaction by applying the energy to the ultraviolet curing resin. The photoinitiator may be composed of an a-hydorxy-based material, a phenylglyoxylate-based material, or a mixture of the two materials.

According to an embodiment of the present invention, the transparent coating layer 150 may include a heat curing resin. The heat curing resin is a raw material which may be dried firmly by heat to allow the transparent coating layer to be a film, thereby providing gloss, scratch prevention, and breakability. The heat curing resin may be a material in which at least one of non-toxic materials such as an epoxy resin, an acrylic resin, and a polyester resin are mixed. In addition, in order to melt a resin in a solid state or adjust viscosity, the heat curing resin may be mixed with one or more of butyl acetate, ethyl acetate, and acetone, and used.

Meanwhile, in another embodiment of the present invention, the gel nail sticker of the present invention may additionally include a waterproof layer.

The thickness of each layer according to an embodiment of the present invention is set to an optimal thickness range in consideration of fit and adhesion, and aesthetic feel caused by the printed layer, and the like.

Meanwhile, the present invention provides a method for manufacturing a gel nail sticker as follows.

In an embodiment of the present invention, the gel nail sticker of the present invention is manufactured by the following manufacturing method.

The method for manufacturing a gel nail sticker includes an adhesive layer forming step S10 of preparing an adhesive composition in which a resin/binder and a solvent are mixed to form an adhesive layer, a print layer forming step S20 of applying a composition containing a pigment for expressing colors on top of the adhesive layer to form a print layer, and a transparent coating layer forming step S30 of forming a transparent coating layer containing a curing resin on top of the print layer.

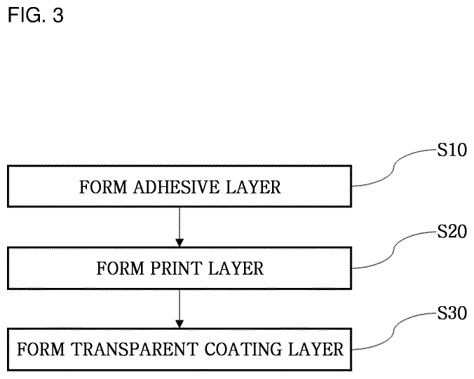

FIG. 3 is a flowchart illustrating one example of the above-described method for manufacturing a gel nail sticker in accordance with still another exemplary embodiment of the present invention.

In an embodiment of the present invention, after the forming of a transparent coating layer S30, curing the transparent coating layer may be further included.

Meanwhile, graphene also has the ability to block ultraviolet light, and thus, may interfere with the curing reaction. Therefore, it is preferable to place the transparent coating layer above the adhesive layer containing graphene.

The curing step goes through an ultraviolet curing step if an ultraviolet curing resin is included, and goes through a heat curing step if a heat curing resin is included. At this time, the ultraviolet curing step may be performed by irradiating ultraviolet light to achieve ultraviolet curing, and the heat curing step is characterized by applying a method of forcibly blowing air maintaining a temperature of approximately 40° C. or higher for approximately 1 minute or more.

Meanwhile, the size of a graphene particle may be approximately 10 nm to 100 nm, preferably approximately 50 nm to 80 nm.

In addition, the thickness of a layered structure constituting the graphene may be approximately 0.35 nm to 1.5 nm.

Hereinafter, preferred embodiments of the present invention will be described in detail to facilitate understanding of the present invention. However, the embodiments are merely illustrative of the present invention, and thus, it will be apparent to those skilled in the art that various modifications and variations can be made without departing from the scope and spirit of the present invention as disclosed in the accompanying claims. It is obvious that such variations and may fall within the scope of the appended claims.

Manufacturing of Gel Nail Sticker

Referring to FIG. 3, the manufacturing method of the present invention includes preparing an adhesive layer containing graphene, coating a composition including a pigment to form a print layer, and forming a transparent layer containing a curing resin. Hereinafter, results showing that various physical properties change depending on the presence or absence of graphene will be examined.

### EXAMPLE 1

A gel nail sticker was manufactured by setting the diameter of graphene particles included in the adhesive layer of the gel nail sticker described above to approximately 30 nm to 40 nm and the content of graphene to approximately 0.01 mass % based on the total mass of the adhesive layer.

### COMPARATIVE EXAMPLE 1

A gel nail sticker was manufactured in the same manner as in Example 1, except that graphene was not contained in the adhesive layer (coated with a composition containing only a resin/binder and a solvent).

### COMPARATIVE EXAMPLE 2

A gel nail sticker was manufactured in the same manner as in Example 1, except that the diameter of graphene particles included in the adhesive layer was approximately 200 nm to 300 nm.

### COMPARATIVE EXAMPLE 3

A gel nail sticker was manufactured in the same manner as in Example 1, except that the content of graphene was excessive at approximately 10 mass % based on the total mass of the adhesive layer.

Anti-Bacterial Performance Test

The anti-bacterial properties of Table 1 below are the values of the anti-bacterial performance test of staphylococcus aureus ATCC 6538 and pseudomonas aeruginosa ATCC 13388 based on the ┌ASTM E 2180:2018┘ standards.

Specifically, gel nail stickers were manufactured according to Examples and Comparative Examples, and then five test subjects were allowed to naturally use the gel nail stickers on their nails for 3 days. Thereafter, the gel nail stickers were peeled off from the nails, and then the anti-bacterial properties thereof were evaluated on the basis of the ASTM E 2180:2018 standards. The figures below are the average numbers of bacteria for five test subjects.

TABLE 1

| | | | Comparative | | Comparative | | Comparative | |
|---|---|---|---|---|---|---|---|---|
| | Example 1 | | Example 1 | | Example 2 | | Example 3 | |
| | Staphylococcus | Pseudomonas | Staphylococcus | Pseudomonas | Staphylococcus | Pseudomonas | Staphylococcus | Pseudomonas |
| | aureus | aeruginosa | aureus | aeruginosa | aureus | aeruginosa | aureus | aeruginosa |
| Initial number | 1.2 × 10⁶ | 1.4 × 10⁶ | 1.3 × 10⁶ | 1.5 × 10⁶ | 1.4 × 10⁶ | 1.5 × 10⁶ | 1.3 × 10⁶ | 1.5 × 10⁶ |
| of bacteria | | | | | | | | |
| (number of | | | | | | | | |
| bacteria/cm²) | | | | | | | | |
| Number of | 1.4 × 10⁴ | 1.5 × 10⁶ | 3.5 × 10⁸ | 4.0 × 10⁸ | 1.8 × 10⁴ | 1.8 × 10⁴ | 1.5 × 10⁴ | 1.6 × 10⁴ |
| bacteria after | | | | | | | | |
| 72 hours | | | | | | | | |
| (number of | | | | | | | | |
| bacteria/cm²) | | | | | | | | |

From the experimental result that the number of bacteria was rapidly increased only in the gel nail sticker of Comparative Example 1 not containing graphene, it is presumed that the presence or absence of graphene has a significant effect on anti-bacterial properties.

Evaluation of Anti-Fungal Degree

The test on five standard published strains of Aspergillus niger ATCC 9642, Chaetomiumglobosum ATCC 6205, Penicillium pinophilum ATCC 11797, Gliocladium virens ATCC 9645 and Aureobasidium pullulans ATCC 15233 was conducted by commissioning FM Research Institute.

TABLE 2

| | | Comparative | Comparative | Comparative |
|---|---|---|---|---|
| | Example 1 | Example 1 | Example 2 | Example 3 |
| Rating of anti- | 1 | 4 | 2 | 1 |
| fungal degree | | | | |

From the result that only the gel nail sticker of Comparative Example 1 not containing graphene has a low rating of anti-fungal degree, it is presumed that the presence or absence of graphene has a significant effect on anti-fungal degree (anti-fungal properties).

Peel Strength Test

When a gel nail sticker is used for a long period of time, an adhesive layer is delaminated, which weakens the durability of the gel nail sticker.

In order to measure peel strength, the size of a specimen should be large. Therefore, specimens having the same components as the gel nail stickers manufactured in Example 1 and Comparative Examples 1 to 3 were prepared. Specifically, specimens having a width of approximately 25.4 mm and a length of approximately 100 mm were specially prepared. The cut specimens were bitten by a jig, and then the peel strength thereof was measured in a 180 degree peeling manner using a tensile strength tester (Hounsfield test equipment, serial No. 0050 model 100 RC). The test was performed under the conditions of a tensile speed of approximately 100 mm/min and a stretch length of approximately 50 mm The peel strength was measured for each specimen a total 5 times, and average values are shown below. It was confirmed that Example 1, which satisfies the technical features of the present invention, had improved peel strength. When the peel strength is improved, there is an advantageous effect in that a gel nail sticker may be used for a long time since the gel nail sticker is not delaminated from nails/toenails even after being used for a long period of time.

TABLE 3

| | | Comparative | Comparative | Comparative |
|---|---|---|---|---|
| | Example | Example | Example | Example |
| | 1 | 1 | 2 | 3 |
| Peel strength | 1.5 | 0.5 | 1.3 | 1.2 |
| (gf/mm) | | | | |

Evaluation of Degree of Restoration of Nail

Gel nail stickers were manufactured according to Examples and Comparative Examples, and then attached on nails for approximately 24 hours. Thereafter, the gel nail stickers were peeled off to observe the shaped thereof. It was confirmed the gel nail sticker containing graphene retained a curved surface of a nail shape, but the gel nail stickers of Comparative Examples became flat again. However, in the case of Comparative Example 3 in which graphene was contained in excess, the nail sticker became flat again, which is presumed to be the result of enhanced physical properties of graphene itself due to the excessive content of graphene.

TABLE 4

| | Evaluation of degree of restoration of |
|---|---|
| | gel nail sticker |
| Example 1 | Bent nail sticker did not become flat |
| Comparative | Bent nail sticker became flat |
| Example 1 | |
| Comparative | Bent nail sticker did not become flat |
| Example 2 | |
| Comparative | Bent nail sticker became flat |
| Example 3 | |

The effect of applying the laminate body of the present invention configured as described above to a gel nail sticker is as follows.

According to the present invention, since graphene particles are contained in the laminate body, it is possible to manufacture a gel nail sticker with improved durability, retention force, and adhesive force, and even when used for a long period of time, bacteria do not grow in the gel nail sticker, allowing the gel nail sticker to be used for a long time.

According to the present invention, it is possible to manufacture a gel nail sticker which may be used for a long period of time due to its excellent durability, and which may be used without causing repulsion since no bacteria growing therein.

Meanwhile, according to the present invention, it is possible to manufacture a gel nail sticker whose number of uses may be reduced due to its excellent durability, and which may not affect the environment due to the eco-friendly properties of graphene even when the gel nail sticker is disposed.

What is claimed is:

1. A gel nail sticker comprising a laminate body, the laminate body including:
an adhesive layer including an adhesive component;
a print layer including a pattern; and
a transparent coating layer including an ultraviolet curing resin which are sequentially laminated in the laminate body,
wherein at least one layer of the laminate body contains graphene.

2. The gel nail sticker of claim 1, wherein the graphene is contained in the adhesive layer.

3. The gel nail sticker of claim 2, wherein the graphene is contained in approximately 0.005 mass % to 0.1 mass % based on the total mass of the adhesive layer.

4. The gel nail sticker of claim 1, wherein the diameter of a particle of the graphene is approximately 10 nm to 100 nm, or approximately 50 nm to 80 nm.

5. The gel nail sticker of claim 1, wherein the adhesive layer contains graphene, a resin, and a binder.

6. The gel nail sticker of claim 1, wherein the transparent coating layer contains an ultraviolet curing resin, wherein the ultraviolet curing resin usually maintains a soft semi-solid state, and when irradiated with ultraviolet light, the ultraviolet curing resin is cured into a polymer material in a solid state by a photopolymerization reaction.

7. A method for manufacturing a gel nail sticker, the method comprising:
an adhesive layer forming step of preparing an adhesive composition in which a resin/binder and a solvent are mixed to form an adhesive layer;
a print layer forming step of applying a composition containing a pigment for expressing colors on top of the adhesive layer to form a print layer; and
a transparent coating layer forming step of forming a transparent coating layer containing a curing resin on top of the print layer,
wherein at least one layer of a laminate body contains graphene.

8. The method of claim 7, wherein the graphene is contained in the adhesive layer.

9. The method of claim 8, wherein the graphene is contained in approximately 0.005 mass % to 0.1 mass % based on the total mass of the adhesive layer.

10. The method of claim 8, wherein the diameter of a particle of the graphene is approximately 10 nm to 100 nm, or approximately 50 nm to 80 nm.
